(19) United States
(12) Patent Application Publication
(10) Pub. No.: US 2003/0022155 A1
(43) Pub. Date: Jan. 30, 2003

(54) Method for detecting hepatitis c virus with hybridomas

(76) Inventors: Agata Budkowska, Clamart (FR); Patrick Maillard, Montlhery (FR); Jadwiga Nitkiewicz, Komorow (PL); Radu Crainic, Jouy En Josas (FR)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(21) Appl. No.: 09/549,685

(22) Filed: Apr. 14, 2000

Related U.S. Application Data
(60) Provisional application No. 60/129,319, filed on Apr. 14, 1999.

(51) Int. Cl. C12Q 1/70; C12N 5/06; C07K 16/08

(52) U.S. Cl. 435/5; 435/339; 530/388.3

(57) ABSTRACT

A method of directly detecting Hepatitis C virus in a fractionated or non-fractionated serum of a patient, by detecting the virus with primers corresponding to viral RNA encoding core protein which said RNA is a light fraction of the total viral RNA, the said light fraction being isolated after ultracentrifugation in a CsCl gradient of human serum containing HCV virus, said light fraction containing most of the circulating infectious HCV virus particles, the method entailing precipitating RNA, then effecting reverse transcription, and then effecting amplification with the primers described herein.

### BACKGROUND OF THE INVENTION

1. Field of the Invention

The present invention relates to a method for detecting hepatitis C virus using hybridomas.

2. Description of the Background

Hepatitis C virus (HCV) is an enveloped positive strand RNA virus, recognized as the major etiologic agent of blood-borne and sporadic non-A, non-B hepatitis. Due to the propensity of this virus to cause chronic infections, and its association with liver cirrhosis and hepatocellular carcinoma, HCV is a significant world-wide health problem.

HCV has a single strand positive sense RNA genome of approximately 9,400 nucleotides in length. The virus has a lipid-containing envelope that is chloroform-sensitive and appears to be necessary for replication. HCV is similar to members of the Flaviviridae family in overall genome organization and in the presumed mechanism of replication. Particularly, HCV genome codes for a single polyprotein precursor of about 3,000 aminoacids that is cleaved into a series of proteins including capsid, two envelope proteins E1 and E2 and 7 putative non-structural proteins some of which are involved the polyprotein processing. Although the entire HCV genome has been sequenced, see Chiron patents: EP 318216, EP 388,232 and PCT WO 90/1443, and the viral proteins and their processing have been well characterized in vitro, little is currently known about the mechanism of HCV infection which leads to viral persistence despite a broad immunological response to viral structural and non-structural proteins. Current diagnoses of HCV infection are based on the detection of viral RNA in serum by polymerase chain reaction (PCR) and antibodies against HCV components by the assays involving multiple HCV recombinant proteins and/or synthetic peptides. However, there are no available diagnostic assays for detection of the structural proteins of circulating virus. The polypeptide composition, antigenic structure of the virion and number of the possible viral serotypes remain unknown.

Putative HCV virion is about 50-60 nm in diameter and is composed of a viral envelope and a 33 nm core. HCV core protein is a highly basic and is mapped to the first 191 aa residues of the HCV polyprotein. This region is well conserved between different HCV isolates and genotypes and shows high degree of homology with nucleocapsid proteins of other flaviviruses. Viral encapsulation requires the self-association and the capacity to interact with the viral RNA. The interaction sites with homologous and heterologous RNA has been mapped to the N-terminal region of the core protein, whereas the main homotypic interaction domain has been mapped to the tryptophan rich aa sequence (73-108). The hydrophobic signal sequence for translocation of El protein into the endoplasmic reticulum is located in the C-terminal part aa (170-191) and is apparently cleaved by proteases associated with cellular membranes at aa 172. Besides its role in viral replication. HCV core protein has many important biological functions, such as modulation of transcription from several cellular promoters , suppression of the HBV gene expression, interaction with the cytoplasmic tail of lymphotoxin receptor and others.

With equilibrium centrifugation and immunoprecipitation studies, it has been demonstrated that HCV populations in serum consist of low density virions associated with P-lipoproteins which are infectious in cultured cells and of the high density fraction that might contain either immune complexes or naked HCV nucleocapsids. Although, several groups have reported the detection of the core antigen by immunological methods in virus-enriched serum samples from HCV-infected individuals after detergent treatment, no free core antigen has yet been isolated from serum and characterized immunochemically.

To date, the two basic tests for HCV are i) PCR, and ii) detection of antibodies in patient serum. However, a need exists for an improved means of detecting HCV.

### SUMMARY OF THE INVENTION

Accordingly, it is an object of the present invention to provide a method of directly detecting HCV in serum of a patient, which represents a surprising improvement over the conventional tests for HCV.

It is also an object of the present invention to provide a method of detecting non-enveloped nucleocapid or non-enveloped core protein of HCV in serum of a patient.

The above objects and others are provided by a method of directly detecting hepatitis C virus in serum of a patient by detecting the virus with primers corresponding to viral RNA encoding core protein which RNA is a light fraction of the total viral RNA, the light fraction being isolated after ultracentifugation in a CsCl gradient of human serum containing HCV virus, the light fraction containing most of the circulating infectious HCV virus particles, which method entails precipitating RNA, effecting reverse transcription and then effecting amplification with the primers described herein.

### BRIEF DESCRIPTION OF THE DRAWINGS

FIG. 1 illustrates HCV-RNA determination by RT-PCR and b-DNA assay.

FIG. 2A illustrates the activity of monoclonal antibody (MAb)VT against HCV core protein.

FIG. 2B illustrates the activity of monoclonal antibody (MAb) 39-72 against HCV core protein.

FIG. 3A illustrates a mapping of epitopes recognized by MAb 39-72 using a panel of synthetic peptides.

FIG. 3B illustrates an epitope analysis of HCV core protein.

FIG. 4 illustrates that MAb VT and MAb 39-72 recognize different, non-overlapping epitopes of HCV core protein.

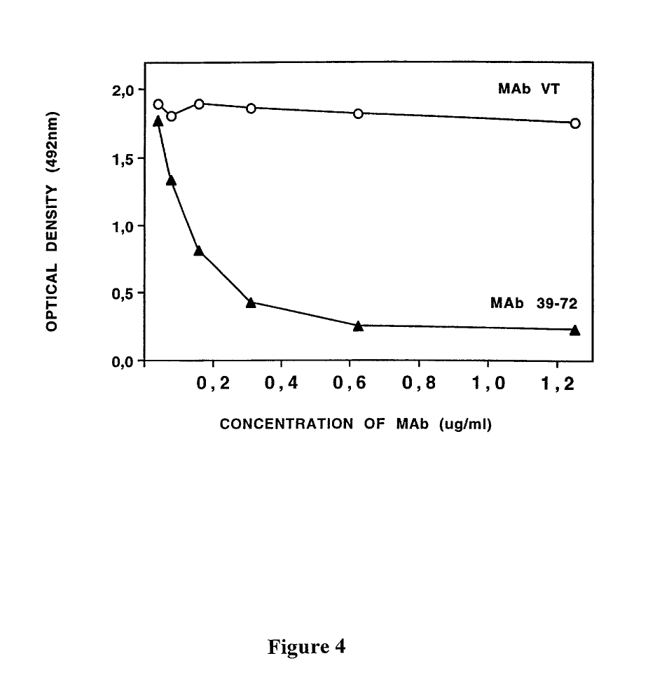

FIG. 5 illustrates the results of ELISA for HCV-core protein.

FIG. 6 illustrates lack of inhibition of MAb 39-72 used in the assay for core antigen by human globulins containing anti-core antibodies.

FIG. 7 illustrates the results of Western-Blot analysis of the anti-HCV-core IgM MAb.

### DETAILED DESCRIPTION OF THE PREFERRED EMBODIMENTS

The present invention provides a surprisingly improved method for detecting HCV. In more detail, the present invention provides a method of detecting HCV by visualization of the presence of core protein by a double sandwich test using at least monoclonal antibodies produced by the hybridoma of the present invention.

Hereinbelow is both a general and detailed description of the present invention involving the detection and immunological characterization of the free core antigen circulating in plasma of HCV carriers. Monoclonal antibodies were prepared by immunization of mice with a natural, serum-derived HCV nucleocapsid and applied for detection of HCV core in serum and liver tissue of HCV infected chimpanzees.

Material and Methods

Measurement of HCV RNA by RT-PCR

HCV RNA in serum and fractions of the gradients was determined by nested polymerase chain reaction (PCR) based on the amplification of the cDNA from the core region of the virus. Viral RNA was isolated using a commercial Rnable reagent from Eurobio. cDNA synthesis and PCR was carried out with amplification using primers according to P. Simmons et al (J. Gen. Virol. 74,661-668, 1993) as detailed below:

RNA was reversely trans-transcribed using a primer sequence 5′-1. CATGTAAGGGTAATCGATGAC, cDNA was amplified using this primer and a primer in the 5′ NCR of the sequence 2. 5′-ACTGCCTGATAGGGTGCTTGCGAG. The second PCR used primers 3. AGGTCTCGTAGACCGTGCATCATG and 4. 5′-TTGCGGGACCTACGCCGGGGGTC.

Detailed RT-PCT Method (RT-PCR Capsid-HCV)

This technique is described according to two protocols. The first one is a normal PCR amplification, but the usual dNTP mix is replaced with a dUTP/dATP, dCTP, dGTP mix in order to perform hydrolysis of the DNA with the Uracil-DNA Glycosylase (UDG) in the event of cross contamination of the PCR products. This hydrolysis step prior to PCR amplification is described in the second protocol. The principle of this hydrolysis step relies on the digestion of DNA matrices that contain dUTP instead of dATP, by the UDG. Only DNA which contains dUTP will be digested. However, precaution which must be taken in UDG use is to check the thermosensibility thereof. Indeed, after digestion of DNA matrix, it is necessary to inactivate the enzyme by heating during at least 5 min. at 95° C. to prevent digestion of the PCR products after amplification.

Antibodies to HCV Core Protein and ELISA Test for Detection of HCV Core Protein

Monoclonal antibody VT directed to the HCV core protein was obtained from Valbiotech (Pads, France); MAb 39-72 was obtained by immunization of mice with a core peptide corresponding to the aminoacids sequence core numbers 39-72.

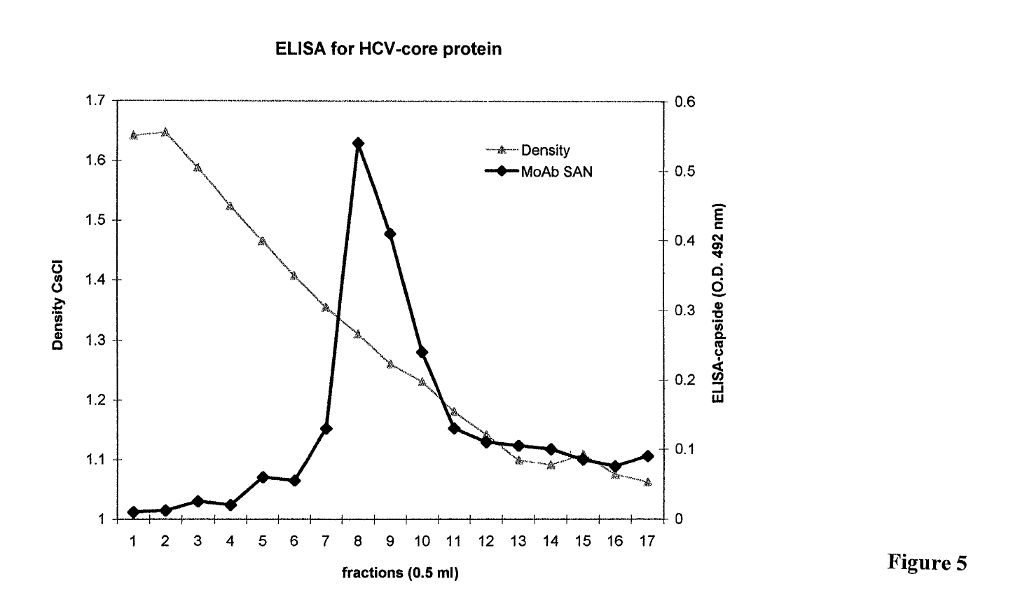

Analysis of the fractions of CsCl gradient with anti-core antibodies revealed the presence of the material reactive with both MAbs VT and 39-72 in fraction, defined as a “heavy fraction”, of a density of 1.32-1.36, much heavier than that of a presumed virion (1.08-1.10 g/ml) defined as the “light fraction”. This observation suggested the presence of non-enveloped nucleocapsids in plasma detectable by ELISA without any treatment. Moreover, the core epitopes detected were apparently exposed and non-covered by human immunoglobulins. Indeed, using inhibition assay no antibodies corresponding to MAb 39-72 could be were detected in a pool of human immunoglobulins prepared from sera with high titer of anti-core antibodies. See FIG. 5.

Two pools of human globulins containing anti-HCV antibodies IGIV and HG were prepared from sera of HCV carriers highly positive for anti-NS3, NS4 and core antibodies by Abbott HCV EIA were kindly provided by Dr. Ali Fattom, Nabi and A. Nowoslawski, respectively.

Western Immunoblotting

Specimens were solubilized in a Tris buffer pH 6.8, containing either 2% SDS or 2% SDS and 5% 2-mercaptoethanol for 2 min at 100° C. Bromheriol blue (0.01%) and 20% sucrose were then added to the samples, the proteins were separated on 12% polyacrylamide gels, and electroblotted to nitrocellulose membranes. The membrane strips were postcoated overnight at 4° C. with 5% skim milk, washed and reacted for 1 h at room temp with monoclonal or polyclonal antibodies diluted in 1% skim milk. HRPO-labeled anti-mouse IgG (heavy+light chains) (Fab)′ fragments, Amersham) of anti-mouse IgM (Sigma) served as a second antibody. After final rinses the blots were visualized with an enhanced chemoluminescence detection system (Amersham).

Epitope Mapping by ELISA

The wells of polyvinyl plates Maxisorb (Nunc, Denmark) were coated overnight at room temp, with 1 μg/ml of synthetic peptides corresponding to different aminoacid sequences of HCV core protein. The plates were washed with PBS containing 0.05% Tween 20 and were blocked 2 h at 37° C. with 3% BSA in PBS containing 0.05% Tween 20. Monoclonal antibodies diluted in PBS were incubated on peptide-coated plated 2 h at 37° C. Following washing as above the wells were incubated with HRPO-labeled anti-mouse IgG (heavy+light chains) (Fab)′ fragments, Amersham) of anti- mouse IgM (Sigma) as a second antibody. The reaction was developed using o-phenylenediamine as the enzyme substrate and the absorbance values were read at 492 nm with an ELISA plates reader.

Competition ELISA With Human and MAbs Anti-HCV Core

Polyvinyl plates were coated with synthetic peptides or purified recombinant proteins in a concentration of 1 μg/ml, blocked and washed as described above. 100 microliters of human globulins prepared from human sera with a high titers of anti-core antibodies or MAbs, fluids were added to the wells and incubated 24 h at 37° C. After washing, peroxidase labeled MAb 39-72 was added to the wells and incubated as before. The plates were developed and read as described above.

Preparation of Mabs to HCV Core Protein

Balb/c mice were immunized intrasplenically with 50 μl of the fraction of CsCl gradient containing HCV core antigen detectable by ELISA. The fraction was dialyzed against PBS and concentrated using Nanosep centrifugal concentrator 300K (Pall Filtron). Three days after immunization mice spleen cells were fused with Sp2/OAg-myeloma cell line. Hybridoma supernatants were screen by ELISA using purified recombinant core protein corresponding to amino acids no. 1 to 120 of the sequence of the nucleocapside. The hybridomas reactive with the recombinant protein were cloned by limiting dilution. The immunoglobulin class of MAbs was determined using anti-mouse IgG (g chain) Amersham and anti-mouse IgM (m chain) (Sigma). The epitope specificity of MAbs was determined using a series of synthetic core peptides.

Results

Fractionation of HCV in Density Gradients

Precipitation of HCV by PEG-6000, previously used for concentration of other viruses allowed the concentration of HCV without lost of viral RNA. Notably, the totality of HCV RNA present originally in the plasma was recovered in the pellet. PEG precipitated preparation was subsequently submitted to ultracentrifugation in sucrose or CsCl gradients. Analysis of distribution of HCV by PCR in sucrose and CsCl gradients after equilibrium centrifugation showed heterogeneity of viral material derived from plasma. The majority of viral RNA was detected in top fractions of a buoyant density of 1.08-1.10 g/ml CsCl and at the density of 1.08 g/ml of sucrose. This RNA could be probably attributed to the b-lipoprotein associated virions since in RNA present in the “light” (top) fractions was stable and could be precipitated 90% with both dextrin sulphate.

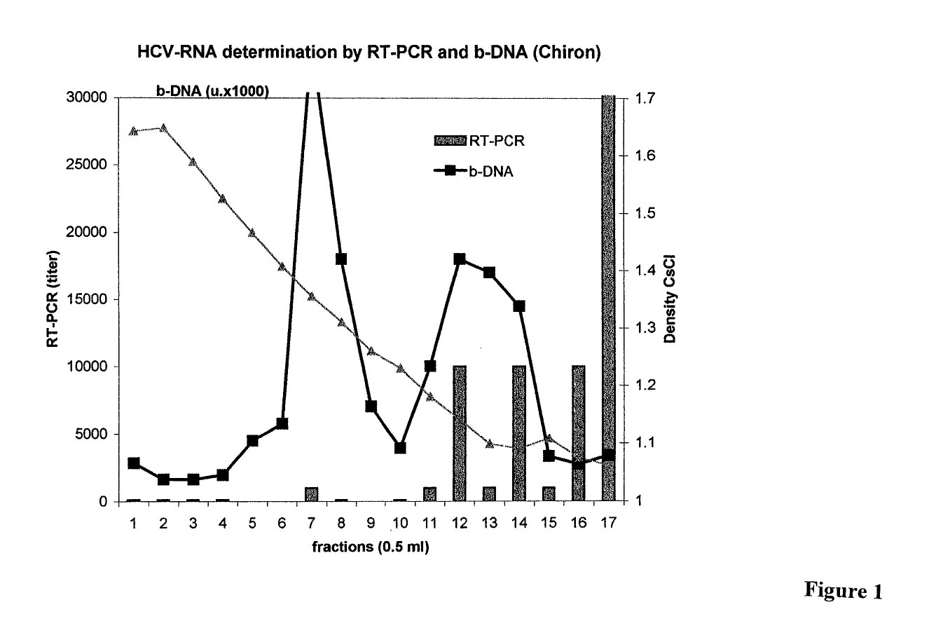

A part of viral RNA was localized in fractions of higher density. See FIG. 1. Interestingly, different profiles of the distribution of viral RNA in the gradient were obtained using routine PCR and a commercial b-DNA assay (Chiron) which apparently does not detect the bulk of viral RNA at the top of the gradient.

Fractionation of HCV Positive Human Plasma

Human plasma (100 ml) from a chronic HCV carrier (voluntary blood donor) seropositive for anti-HCV antibodies and containing HCV of 1a genotype (titre 10-5 by PCR) was stored at 80° C. The plasma was thawed and clarified 10 min at 10,000 rpm, PEG 6000 was then added to the clarified plasma to a final concentration of 10% and NaCl to a final concentration of 0.4 M. The mixture was incubated overnight at 4° C. and precipitated virus separated by centrifugation for 1 h at 11,000 rpm in rotor of a Centrikon centrifuge. The pellet, was resuspended in a 13 ml of a 0.01 M Tris-HCl pH 7.2 containing 0.15 M NaCl. The pellet was subjected to centrifugation in a discontinuous CsCl gradient (1.10-1.60; g/ml 1.5 ml of each solution) prepared in PBS and containing protease inhibitors-1 mM PMSF, 2 μg/ml aprotinine and 10 mM EDTA. Centrifugation was carried out in a Beckman SW 41 rotor 48 h at 40,000 rpm. Fractions (1 ml) were collected from the bottom of the tube and assayed for HCV RNA by PCR and for the presence of core antigen by ELISA.

Detection of HCV Core Antigen in Fractions of CsCl Gradient by ELISA

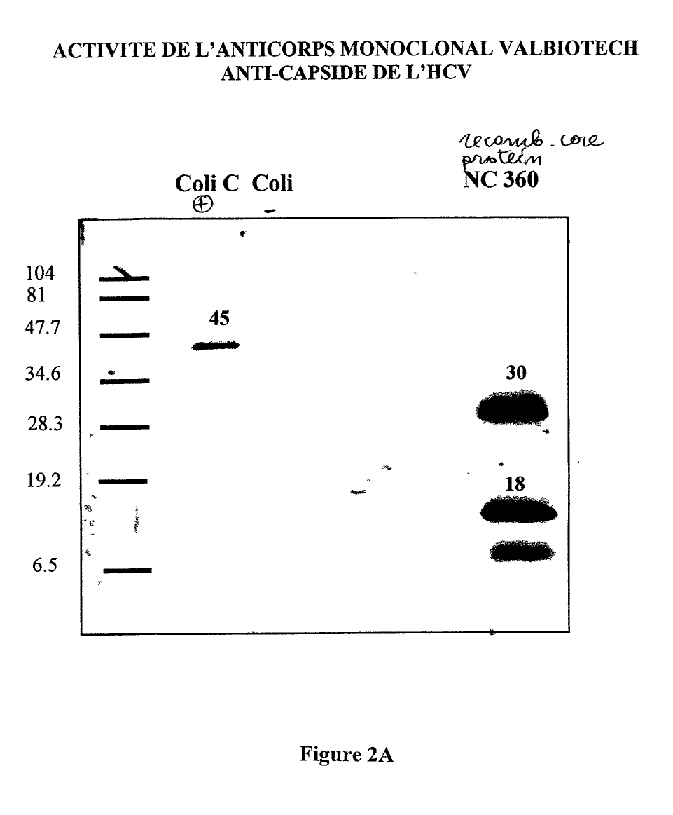

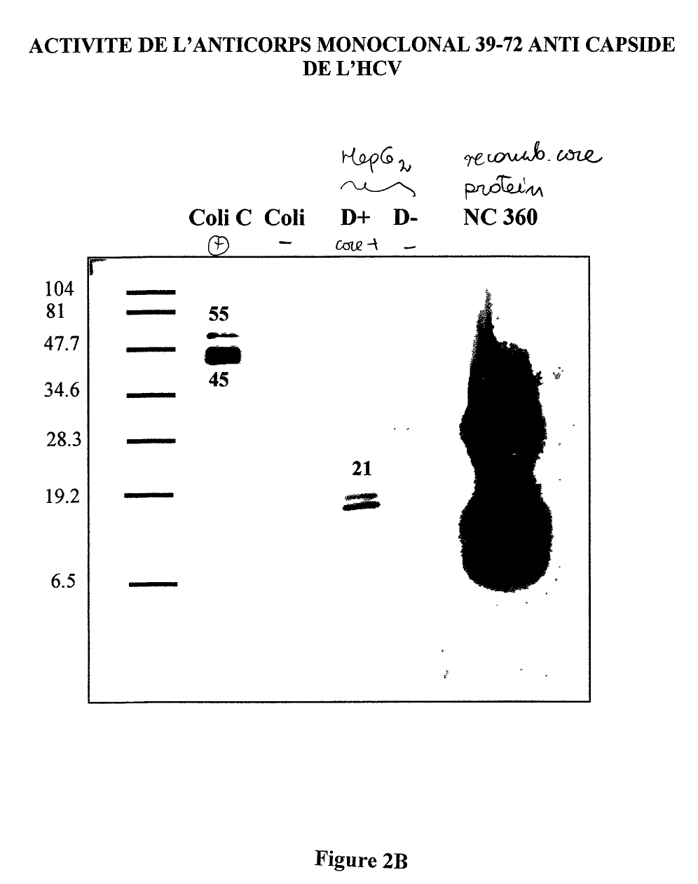

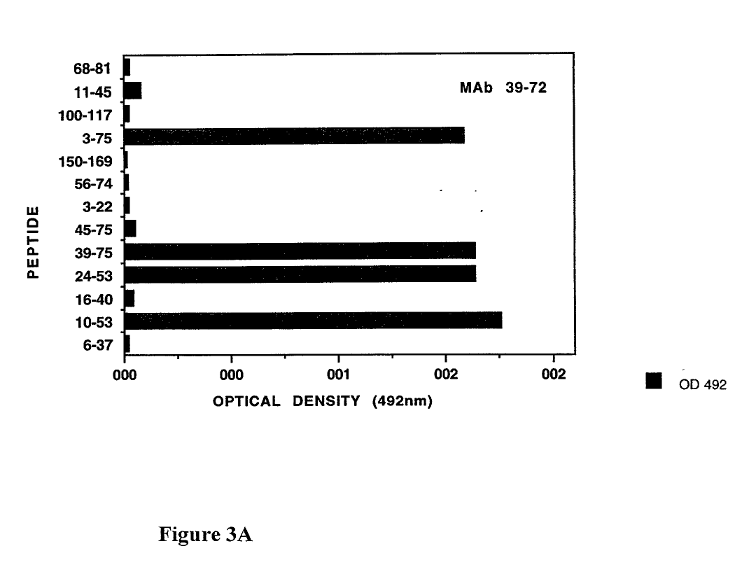

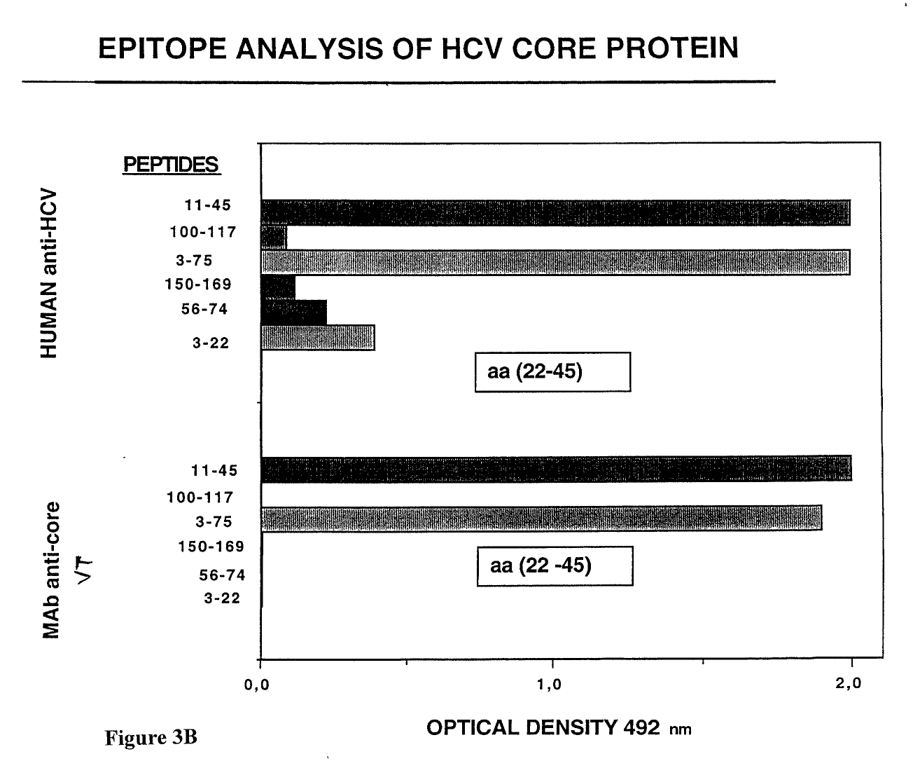

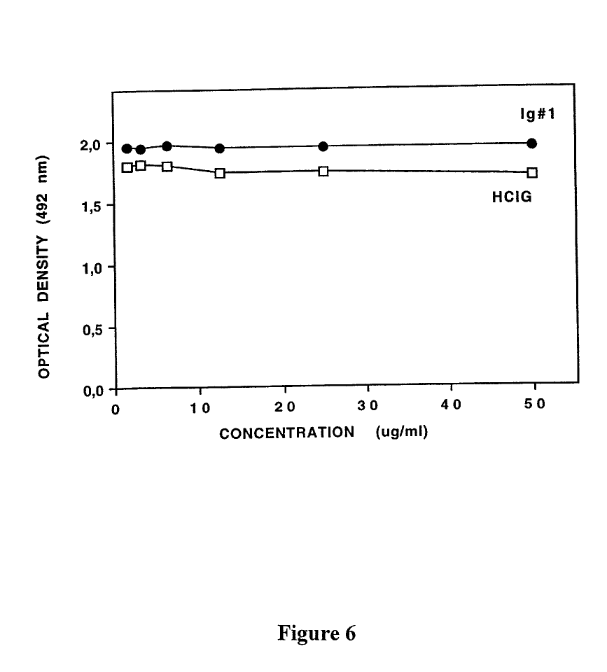

Two MAbs, designated as MAb VT (Valbiotech, Paris, France, immunizing antigen non-communicated by the producer) and MAb 39-72, obtained by immunization of mice with a core peptide corresponding to the aminoacids sequence core numbers 39-72 were used for the development of the assay for detection of the HCV core protein. See FIGS. 2A and 2B. The specificity of these monoclonal antibodies was ascertained by Western blot with recombinant HCV core proteins and epitopes recognized by these MAbs were delineated using a series of synthetic peptides encompassing HCV core protein: MAb VT was reactive with the epitope located in the aminoacid sequence 24-37, and MAb 39-72 was reactive with an epitope located in the aa sequence 40-54. See FIGS. 3A-3B. The competitive binding assay confirmed that these two MAbs recognized two different, non-overlapping and non-adjacent epitopes. See FIG. 6.

To exclude the possibility of the interference of rheumatoid factor (RF) or other non-specific reactivity with the detection of the core antigen, the presence of RF in the gradient was tested. The RF reactivity was detected by latex test in parallel with the non-specific binding to a control (unrelated to HCV) in the fractions of the gradient located at the lower density than that of the core activity.

To evidence that, in fact, core antigen was present in the gradient, fractions exhibiting the core antigenicity were polled, concentrated by dialysis in the Nanosep centrifugal concentrator 300K (Pall Filtron). 300,000 kda and injected to Balb/c mice to produce MAbs. The hybridomas were selected by ELISA with synthetic 1-130 peptide and subsequently tested with a series of overlapping peptides corresponding to different regions of the core antigen. According to these results, it was deduced that the obtained MAb recognized a linear epitope which is localized in the aminoacid sequence (45-75) of the core region.

The development of an effective vaccine against HCV is important, but is rendered difficult because of the variability of the virus and unknown antigenic structure of the virion. Identification of the epitopes conserved among different HCV genotypes would be of importance for future development of the immunological assays for detection of the HCV proteins in serum.

The physical properties of HCV particles have been analyzed by ultracentrifugation in sucrose gradients by several groups. Two main populations of HCV particles according to their floating density were found in sera of patients with chronic HCV infection: low-density virus particles (1.06-1.12 g/ml) and high density virus particles (1.18-1.21 g/ml). Virus particles with high density has been apparently associated with immunoglobulins or was supposed to represent partially or completely naked nucleocapsids Kanto. The virus particles of low density were not associated with immunoglobulins, and accumulated base changes in the hyper variable region of the E2 envelope domain of the genome. Changes in the relative proportions of these viral populations have been observed. Kanto and Hino. The increase of the relative numbers of the high density virions correlated with the disease activity and heterogeneity in HVR1 region, whereas patients with a predominance of the low density fraction showed sustained response to interferon treatment.

Core antigen has been detected in by use of monoclonal antibodies after treatment of serum concentrates with detergents or denaturing agents. Tak, Tanaka, Kashiwakuma, Orito, and Takahashi. The core antigen was detected in sera of non-responders to IFN-a but not in patients with a sustained response and was correlated to the level of viral RNA. Tanaka. Isolated nucleocapsid-like HCV particles were observed by electron-microscopy (EM) of the detergent-treated, RNA rich fractions. Taka. Few reports suggested the presence of naked, unenveloped HCV nucleocapsids in sera of HCV carriers which could be observed by EM Trest, or detected in serum by Mabs. Kanto and Maslowa. However this population of HCV has not yet been isolated and characterized immunochemically.

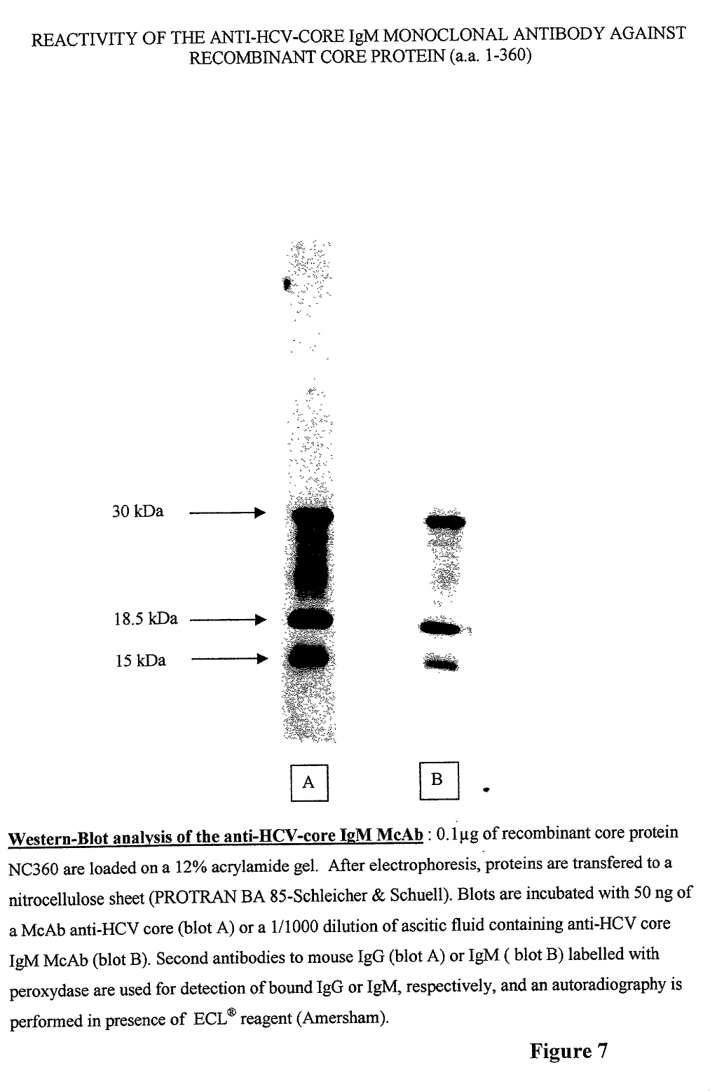

In the following experiment using well-characterized MAbs, the core epitopes exposed on the native nucleocapsid protein were detected in serum. These monoclonal antibodies recognized the non-overlapping epitopes of the HCV core, located close to each other in the aminoacid sequence 24-53. Since reactive with MAbs, these epitopes were not covered by human anti-core antibodies and no corresponding specificity could be detected in a pool of antibodies from chronic HCV carriers. The core antigen was isolated from serum and was shown to be immunogenic in mice. MAbs obtained by immunization with a native serum-derived core protein bound to the linear epitope located in the aa sequence (45-68) as evidenced with synthetic peptides and recognized recombinant cone protein in Western blot. See FIG. 7. This epitope is conserved between different HCV genotypes and is adjacent to the epitopes recognized by the MAb 39-72 used for detection of the core antigen in plasma.

MAbs raised against the natural core antigen was used to detect HCV core antigen in a liver tissue of chronically infected chimpanzee. This MAb represents a new reagent for the study of HCV biology and for the immunological detection of the viral antigen in sera of patients with HCV infection.

### Protocols Used

I-RNA Extraction From Serum

In a 1.5 ml Eppendorf tube, extract 100 μl, 10 μl and 1 μl of each serum sample. Add respectively 0 μl, 90 μl or 99 μl of sterile water (qsp 100 μl).

Add 1 ml of RNable® (Eurobio). Mix 20 sec. and let 5 min. on ice.

Add 100 μl (1/10^{th} vol.) CHCl₃ (ReadyRed, Appligene), mix and centrifuge 10 min. at 14000 rpm. Save the colorless supernatant in a new tube.

Add 500 μl of CHCl₃, mix and centrifuge 10 min.

Save the supernatant (#500 μl) and add 50 μl 3M NaOAc pH 5.2, 2 μl of SeeDNA™ (Amersham, RPN 5200) and proceed to an ethanol precipitation with 2 vol. (1 ml) of 100% ethanol. Mix, and centrifuge 10 min. at 1400 rpm at 4° C.

Wash the RNA pellet with 1 ml of cold 70% ethanol. Centrifuge 10 min.

Remove all the supernatant, dry the walls of the tube with a Kimwipes® and resuspend the RNA pellet with 20 μl of water containing 2 mM DTT and 2 U/μl Rnasin.

Store at −80° C.

I-cDNA Synthesis (Common to Both Protocols)

In a 500 μl Eppendorf tube, add: (final conc.)

5 μl of purified RNA

5 μl of water

and 1 μl reverse-sense primer SIM 2R.

Recover the mix with one drop of mineral oil, centrifuge briefly and place on the thermocycler for 10 min. at 70° C. and immediately on ice. Centrifuge before the addition of 14 μl of the following mix:

5 μl reverse-transcription buffer 5X (1X)

1.25 μl dNTP 10 mM (0.5 mM)

0.5 μl DTT 100 mM (2 mM)

1 μl RNase inhibitor 40 U/μl (1.6 U/μl)

1.25 MMLV 200U/μl (250 U)

5 μl H₂O (qsp 25 μl)

Centrifuge briefly before incubation 1 hour at 37° C. Inactivate the RT during 10 min. at 95° C. and dip the tubes on ice. At this step, the cDNA can be kept at −80° C.

II-PCR Amplification

| A1 - Outer PCR without UDG | | A2 - Outer PCR with UDG | |
|---|---|---|---|
| hydrolysis | | hydrolysis | |
| Prepare a mix of these components | | Prepare a mix of these components | |
| in a 1 ml Eppendorf tube on ice | | in a 1 ml Eppendorf tube on ice | |
| (final conc.): | | (final conc.): | |
| 5 | μl buffer 10X | 5 | μl buffer 10X |
| 1.5 | μl MgCl₂ 50 mM (1.5 mM) | 1.5 | μl MgCl₂ 50 mM (1.5 mM) |
| 2.5 | μl dUTP/NTP mix 4 mM | 2.5 | μl dUTP/NTP mix 4 mM (0.2 |
| | (0.2 mM) | | mM) |
| 1 | μl reverse sense primer | 1 | μl sense primer SIM 1S |
| | SIM 2R (50 pmole) | | (50 pmole) |
| 33.5 | μl H₂O (qsp 50 μl) | 1 | μl reverse sense primer |
| 0.5 | μl Eurobiotaq (2.5 U) | | SIM 2R (50 pmole) |
| | | 32.5 | μl H₂O (qsp 50 μl) |
| | | 0.5 | μl UDG (0.5 U) |
| | | 0.5 | μl Eurobiotaq (2.5 U) |

Dispense 45 μl of this mix in each thin-walled PCR tube on ice. Add a drop of mineral oil and close the caps.

Under the hood: add 5 μl of cDNA, centrifuge the tubes briefly and put them:

Under the hood: add 5 μl of cDNA, centrifuge the tubes
briefly and put them:

| in the thermocycler block once the | | at 37° C. during 15 min. | |
|---|---|---|---|
| temperature has reached at | | and then, denature the UDG | |
| least 80° C. (Program No. 6) | | during 5 min. at 85° C. | |
| | | and 10 min. at 95° C. | |
| | | before starting the | |
| | | amplification (Program No. 5) | |
| Amplification cycles (Prog. 6): | | Amplification cycles (Prog. 5): | |
| First Cycle: | 94° C.-5 min. | First Cycle: | 85° C.-5 min. |
| | 50° C.-1 min. | | 95° C.-10 min. |
| | 72° C.-1 min. | | 50° C.-1 min. |
| | | 72° C.-1 min. | |
| 25 cycles: | 94° C.-50″ | 25 cycles: | 94° C.-50″ |
| | 55° C.-50″ | | 55° C.-50″ |
| | 72° C.-50″ | | 72° C.-50″ |
| Elongation: | 72° C.-10 min. | Elongation: | 72° C.-10 min. |
| Stop: | 4° C.-5 min. | Stop: | 4° C.-5 min. |

Note: At the end of this application it is important to centrifuge the tubes before opening to avoid contamination and to perform the nested amplification immediately.

B—Inner PCR

This step is common to both protocol because the first amplification product must not be digested by UDG.

Prepare a mix of these components in a 1 ml Eppendorf tube on ice:

5 μl buffer 10X

1.5 μl MgCl₂ 50 mM (1.25 mM)

2.5 μl dUTP/NTP mix 4 mM (0.2 mM)

1 μl internal sense primer SIM 3S (50 pmole)

1 μl internal reverse sense primer SIM 4R (50 pmole)

33.5 μl H₂O (qsp 50 μl)

0.5 μl Eurobiotaq (2.5 U)

Dispense 45 μl of this mix in each thin-walled 0.5 ml PCR tubes on ice. Add a drop of mineral oil.

Under the hood: add 5 μl of DNA, centrifuge and put the tubes on the PCR block once the temperature has reached at least 80° C.

| | | |
|---|---|---|
| Amplification cycles: | First cycle: | 94° C.-5 min. |
| (Program No. 6) | | 50° C.-1 min. |
| | | 72° C.-1 min. |
| | 25 Cycles: | 94° C.-50″ |
| | | 55° C.-50″ |
| | | 72° C.-50″ |
| | Elongation: | 72° C.-10 min. |
| | Stop: | 4° C.-5 min. |

Note: At the end of the amplification it is important to centrifuge the tubes before opening to avoid contaminations and to analyze the products immediately or maintain them at −20° C.

Preparation of dUTP/dNTP-mix

| | |
|---|---|
| Stock solutions: | 250 μl - 20 mM solution dUTP (Epicentre/TEBU) |
| | 25 μM - 100 mM dUTP solution (USB/Amersham) |
| | dNTP 25 μM - 100 mM solutions kit Pharmacia |
| Preparation: | A-TEBU 20 mM dUTP: |
| | Dilute 1/25 the 100 mM solutions of the dATP, |
| | dCTP and cGTP (4 mM final) |
| | Dilute the Epicentre/TEBU dUTP 20 mM |
| | solution 1/4 to get a 5 mM solution. Mix 1 vol. |
| | of each dNTP diluted solution to get the 4 mM |
| | solution of dUTP/dNTP mixture. |

B-USB 100 mM dUTP: In a 1.5 ml tube, add 40 μl of each dATP, dCTP and dGTP 100 mM stock solutions (Pharmacia) and 50 μl of the 100 mM dUTP stock solution (USB). Complete to 1 ml (830 μl) with sterile water to obtain the 4 mM solution of dUTP/dNTP mixture.

Preparation of the Solution to Resuspend RNA Pellets

930 μl pure sterilized water

20 μl 0.1 M DTT

50 μl Rnasin

Analysis of the Distribution of HCV RNA by RT-PCR and B-DNA In CsCl Gradient

The majority of viral RNA was detected by RT-PCR in top fraction (“light fraction”) of the gradient corresponding to buoyant density of 1.06-1.10 g/ml CsCl. According to the literature (and also our observation that the majority HCV RNA detectable by RT-PCR can be precipitated with dextran sulfate) this part of RNA could be attributed to the HCV virions associated to β-lipoproteins.

Only a minor part of viral RNA was detected by RT-PCR in fractions of higher density; in contrast b-DNA assay which was much more effective at higher density range and two peaks of HCV RNA could be detected by this assay at 132-36 and the second at 1.10-1.15 g/ml. Moreover, the peak of RNA detected by b-DNA at the density of 1.32-1.36 g/ml corresponded to the localization of the core antigen by ELISA.

The hybridoma described in the present application ws deposited at the C.N. C.M. in France on Apr. 14, 1999, under accession number 1-2183.

Having described the present invention, it will now be apparent that many changes and modifications may be made to the above-described embodiments without departing from the spirit and the scope of the present invention.

Finally, attached to and incorporated into this disclosure are copies of the following publications:

1) Journal of General Virology, 74, 661-668 (1993), Simmond. et al;

2) “Detection et Caracterisation de la Nucleocapside du Virus de L'Hepatite C (VHC) Dans le Serum des Patients Infectes”, Mailard, P. et al;

3) “Analyse de la Structure Antigenique de Virus De L'Hepatite C (VHC)”, Budkowska et al;

4) Archives of Virology, “Ultrastructural and physicochemical characterization of the hepatitis C virus recovered from the serum of an agammaglobulinemic patient,” 143:2241-2245 (1993), Trestard et al;

5) Journal of Medical Virology, “Detection of Hepatitis C Virus Core protein Circulating Within Different Virus particle Populations,” 55:1-6 (1998), Masalova et al.

Listed below of additional are citations for additional background publications.

### REFERENCES

### Background

Hihahata M., Shimizu Y. K., Kato H., et al., “Equilibrium Centrifugation Studies of Hepatitis C Virus: Evidence for circulating Immune Complexes”, J. Virol., 67, 1953-1958, 1993;

Koshy, R. L., Inchauspe, G., “Evaluation of Hepatitis C Virus protein Epitopes for Vaccine Development, Trends in Biotechnology, 14, 364-369, 1996;

Thomssen, R., Bonk, S., Propfe C., Heerman, K. H., Kochel H. G., Uy, A., “Association of Hepatitis C Virus in Human Sera with β-lipoprotein”, Med. Microbiol. Immunol, 181, 293-300, 1992;

Takahashi K., Okamoto H., Kishimoto S., Munekata E., Tachibana K., Akahane Y., Yoshizawa H., and Mishiro S., “Demonstration of a Hepatitis C Virus Specific Antigen Predicted from the Putative Core Gene in the Circulation of Infected Host,” J. Gen. Virol., 73 667-672, 1992;

Takahaski, K., Kishimoto, S., Yoshizawa, H., Okamoto, H., Yoshikawa, A., and Mishiro, S., “p26 protein and 33 nm Particle associated with nucleocapsid of hepatitis C Virus Recovered from the Circulation of Infected Host,” Virology, 191, 431-434, 1992.

What is claimed is:

1. A set of primers selected from the group consisting of (1) and (2); and (3) and (4):
1: CAT(or C).GTA (or G).AGG.GTA.TCG.ATG.AC, and
2: ATC.GCC.TGA.TAG.GGT.GCT.TGC.GAG; and
3: AGG.TCT.CGT.AGA.CCG.TGC.ATC.ATG, and
4: TTG.CG.G(or T)G (or C).A.CCT.A(or T)CG.CCG.GGG.GTC.

2. A method of directly detecting Hepatitis C virus in a fractionated or non-fractionated serum of a patient by detecting said virus with primers corresponding to viral RNA encoding core protein, said RNA being a light fraction of the total viral RNA, said light fraction being isolated after ultracentrifugation in a CsCl gradient of human serum containing HCV virus, and containing most of the circulating infectious HCV virus particles, the process comprising precipitating RNA, then effecting reverse transcription, and then effecting amplification with the set of primers of claim 1.

3. A method of detecting non-enveloped nucleocapsid or non-enveloped core protein of HCV in serum of patients, which comprises:
a) contacting the native serum of patients, optionally treated by chemicals or by physical process or fractionation, with monoclonal antibodies recognizing core protein or nucleocapsid protein;
b) eliminating compounds of the reaction not forming an immune complex with the monoclonal antibodies and which undergo non-specific reaction; and
c) detecting the immune complex formed between the core protein or the nucleocapsid protein and a monoclonal antibody by adding a second monoclonal antibody recognizing the core protein, the second monoclonal antibody being labeled by a radioactive or a non-isotopic marker.

4. The method of claim 3, wherein said non-isotopic marker is a fluorescent or enzymatic marker.

5. The method of claim 3, wherein the serum of the patient is fractionated by:
a) precipitating serum by PEG (polyethyleneglycol);
b) centrifuging the pellet after precipitation containing the HCV virus; resuspending the same in a buffer;
c) ultra-centrifuging the viral suspension in a CsCl gradient (cesium chloride gradient) optionally containing proteases inhibitors; and
d) testing each fraction.

6. The method of claim 3, wherein in step b), said compounds not forming said immune complex or forming non specific complex comprise rheumatoid factor.

7. A diagnostic kit for HCV infection, comprising at least:
a) a solid phase on which antibodies against nucleocapsid or core protein are coated;
b) a sample of purified core protein or nucleocapsid protein of HCV;
c) a sample of monoclonal antibodies labeled and unlabeled specific of HCV protein and, if necessary, a substrate to reveal the marker;
d) a sample of negative human serum;
e) a sample of positive control serum; and optionally
f) buffers and chemicals for testing said serum.

8. A purified polyclonal or a purified monoclonal antibody, which reacts with epitopes of core protein of HCV and which are not bound by human antibodies in the serum of patients and which react with the nucleocapsid or the core protein in infected tissues.

9. A hybridoma deposited at the C.N.C.M. on Apr. 14, 1999, under accession number 1-2183.

10. The process of claim 3, which comprises:
a) separating nucleocapsid or core protein from the remaining compounds present in the serum which interfere with the detection of the nucleocapsid in tissues or in serum; and
b) detecting the same with polyclonal or monoclonal antibodies recognizing the non-enveloped antigens of HCV.
